(19) United States
(12) Patent Application Publication
(10) Pub. No.: US 2025/0387606 A1
(43) Pub. Date: Dec. 25, 2025

(54) Latch Mechanism for Closed System Transfer Device

(71) Applicant: Becton, Dickinson and Company, Franklin Lakes, NJ (US)

(72) Inventor: Sze Jiunn Ang, Singapore (SG)

(21) Appl. No.: 19/243,082

(22) Filed: Jun. 19, 2025

Related U.S. Application Data
(60) Provisional application No. 63/661,952, filed on Jun. 20, 2024.

(51) Int. Cl. A61M 39/10; A61M 39/00

(52) U.S. Cl. CPC A61M 39/1011; A61M 2039/0009; A61M 2039/1027; A61M 2039/1066; A61M 2205/3331

(57) ABSTRACT

A system for transferring fluids, including a syringe adapter having a housing defining a first end, a second end, and a longitudinal axis extending from the first end to the second end; a cannula positioned within the housing; a seal arrangement movably positioned within the housing; at least one locking element extending from the housing; and a vial adapter releasably attachable to the syringe adapter, the vial adapter including a body with a first end and a second end opposite the first end, wherein the first end is positionable within the housing of the adapter and the second end is configured to attach to a container; and at least one retention element coupled to the body, the retention element configured to rotationally receive the at least one locking element to restrict axial movement of the syringe adapter.

### CROSS-REFERENCE TO RELATED APPLICATION

The present application claims priority to U.S. Provisional Patent Application No. 63/661,952 entitled “Latch Mechanism for Closed System Transfer Device” filed Jun. 20, 2025, the entire disclosure of which is hereby incorporated by reference.

### BACKGROUND OF THE INVENTION

### 1. Field of Art

The present disclosure relates generally to a system for the closed transfer of fluids. More particularly, the present disclosure relates to a system that provides leak-proof scaling and pressure equalization during engagement of a cannula with a vial, during transfer of a substance from a vial chamber to a barrel chamber via the cannula, and during disengagement of the cannula from the vial.

### 2. Description of Related Art

Health care providers reconstituting, transporting, and administering hazardous drugs, such as cancer treatments, can put health care providers at risk of exposure to these medications and present a major hazard in the health care environment. For example, nurses treating cancer patients risk being exposed to chemotherapy drugs and their toxic effects. Unintentional chemotherapy exposure can affect the nervous system, impair the reproductive system, and bring an increased risk of developing blood cancers in the future. In order to reduce the risk of health care providers being exposed to toxic drugs, the closed transfer of these drugs becomes important.

Some drugs must be dissolved or diluted before they are administered, which involves transferring a solvent from one container to a sealed vial containing the drug in powder or liquid form, by means of a needle. Drugs may be inadvertently released into the atmosphere in gas form or by way of aerosolization, during the withdrawal of the needle from the vial and while the needle is inside the vial if any pressure differential between the interior of the vial and the surrounding atmosphere exists.

### SUMMARY OF THE INVENTION

The present disclosure provides a system for transferring fluids, including: a syringe adapter comprising: a housing defining a first end, a second end, and a longitudinal axis extending from the first end to the second end; a cannula positioned within the housing; a seal arrangement movably positioned within the housing; at least one locking element extending from the housing; and a vial adapter releasably attachable to the syringe adapter, the vial adapter including: a body with a first end and a second end opposite the first end, wherein the first end is positionable within the housing of the adapter and the second end is configured to attach to a container; and at least one retention element coupled to the body, the retention element configured to rotationally receive the at least one locking element to restrict axial movement of the syringe adapter. The body of the vial adapter may define a lumen therein, and the vial adapter may include a membrane adjacent the first end of the body in fluid communication with the lumen. The membrane may be positionable adjacent the seal arrangement of the syringe adapter when the first end of the vial adapter is positioned within the housing of the syringe adapter. At least a portion of the cannula may be positioned within the lumen when the membrane is positioned adjacent the seal arrangement of the syringe adapter.

The second end of the vial adapter may include a spike member having a fluid channel therein in fluid communication with the lumen. The spike member may include a vent channel in fluid communication with a pressure equalization device. The pressure equalization device may include a balloon. The syringe adapter may include a spring configured to bias a position of the seal arrangement within the housing.

The at least one locking element may extend from the housing transverse to the longitudinal axis. The at least one locking element may be adjacent the second end of the housing, and the first end of the housing may include a luer connector defining a passageway therethrough in fluid communication with the cannula. The at least one locking element may include a plurality of locking elements spaced equidistant around a circumference of the housing. The at least one retention element may define a slot transverse to the longitudinal axis. The at least one retention element may include a plurality of retention elements spaced equidistant around a circumference of the body, and at least a portion of the housing of the syringe adapter may be positionable between the plurality of retention elements.

A system for transferring fluids is disclosed, including a syringe adapter comprising: a housing defining a first end, a second end, and a longitudinal axis extending from the first end to the second end; a cannula positioned within the housing; a seal arrangement movably positioned within the housing; a plurality of locking elements extending from an exterior of the housing transverse to the longitudinal axis; and a vial adapter releasably attachable to the syringe adapter, the vial adapter including: a body with a first end and a second end opposite the first end, wherein the first end is positionable within the housing of the adapter and the second end is configured to attach to a container; the body defining a lumen therein; a spike member at the second end, the spike member having a fluid channel in fluid communication with the lumen; and a plurality of retention elements coupled to the body, each retention element defining a slot therein configured to rotationally receive at least one of the plurality of locking elements and restrict axial movement of the syringe adapter.

The vial adapter may include a membrane sealing at least a portion of the lumen, and the membrane may be positionable adjacent the seal arrangement of the syringe adapter when the first end of the vial adapter is positioned within the housing of the syringe adapter. At least a portion of the cannula may be positioned within the lumen when the membrane is positioned adjacent the seal arrangement of the syringe adapter. The spike member may include a vent channel in fluid communication with a pressure equalization device. The plurality of locking elements may be spaced equidistant from each other adjacent the second end of the housing.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings, wherein:

FIG. 1 is a perspective view of an example of a syringe adapter of a fluid transfer system constructed in accordance with the principles of the present disclosure;

FIG. 2 is a cross-sectional view of the syringe adapter of FIG. 1;

FIG. 3 is a perspective view of an example of a vial adapter of a fluid transfer system constructed in accordance with the principles of the present disclosure;

FIG. 4 is a cross-sectional view of the vial adapter of FIG. 3;

FIG. 5 is a perspective view of an example of a partially assembled system with the syringe adapter of FIG. 1 and the vial adapter of FIG. 3;

FIG. 6 is another perspective view of the system of FIG. 5;

FIG. 7 is another perspective view of the system of FIG. 5 with the syringe adapter of FIG. 1 secured to the vial adapter FIG. 3; and

FIG. 8 is a cross-sectional view of the assembled system of FIG. 7.

### DETAILED DESCRIPTION OF THE INVENTION

*The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As used herein, the singular form of “a”, “an”, and “the” include plural referents unless the context clearly dictates otherwise.

Spatial or directional terms, such as “left”, “right”, “inner”, “outer”, “above”, “below”, and the like, relate to the embodiments or aspects as shown in the drawing figures and are not to be considered as limiting as the embodiments or aspects can assume various alternative orientations.

All numbers used in the specification and claims are to be understood as being modified in all instances by the term “about”. By “about” is meant within plus or minus twenty-five percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less. The ranges and/or ratios disclosed herein represent the average values over the specified range and/or ratio.

The terms “first”, “second”, and the like are not intended to refer to any particular order or chronology, but refer to different conditions, properties, or elements.

All documents referred to herein are “incorporated by reference” in their entirety.

The term “at least” is synonymous with “greater than or equal to.”

As used herein, “at least one of” is synonymous with “one or more of.” For example, the phrase “at least one of A, B, or C” means any one of A, B, or C, or any combination of any two or more of A, B, or C. For example, “at least one of A, B, and C” includes A alone; or B alone; or C alone; or A and B; or A and C; or B and C; or all of A, B, and C.

The word “comprising” and “comprises”, and the like, does not exclude the presence of elements or steps other than those listed in any claim or the specification as a whole. In the present specification, “comprises” means “includes” and “comprising” means “including.”

As used herein, the terms “parallel” or “substantially parallel” mean a relative angle as between two objects (if extended to theoretical intersection), such as elongated objects and including reference lines, that is from 0° to 5°, or from 0° to 3°, or from 0° to 2°, or from 0° to 1°, or from 0° to 0.5°, or from 0° to 0.25°, or from 0° to 0.1°, inclusive of the recited values.

As used herein, the terms “perpendicular”, “transverse”, “substantially perpendicular,” or “substantially transverse” mean a relative angle as between two objects at their real or theoretical intersection is from 85° to 90°, or from 87° to 90°, or from 88° to 90°, or from 89° to 90°, or from 89.5° to 90°, or from 89.75° to 90°, or from 89.9° to 90°, inclusive of the recited values.

The present disclosure provides a system that provides leak-proof sealing and pressure equalization during engagement of a cannula with a vial, transfer of substances and/or fluids therethrough, and during disengagement of the cannula from the vial. Referring now to the figures in which like reference designations refer to like elements, examples of such a closed fluid transfer system 10 generally including a syringe adapter 12 and a vial adapter 14 operable to securely transfer fluids is shown. The system 10 provides substantially leak-proof sealing during transfer of a fluid from a first container 18, such as a vial, to a second container (not shown), such as a syringe, IV bag, or patient IV line. The leak-proof sealing of the system 10 substantially prevents leakage of both air and liquid during use of the system 10. Although not shown, the system 10 may further include an IV bag adapter as well as other components typically utilized in closed system transfer devices, such as infusion lines and extension sets.

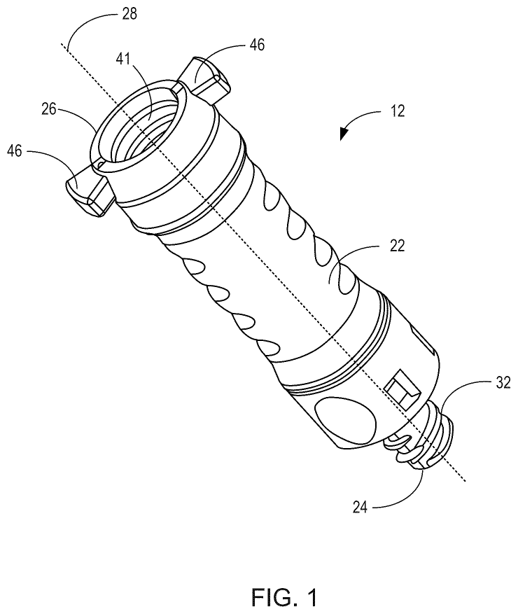

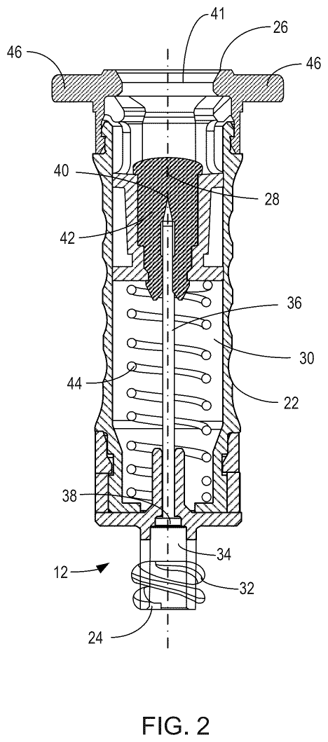

Referring to FIGS. 1 and 2, an example of the adapter 12 is illustrated. The syringe adapter 12 is configured to receive at least a portion of the vial adapter 14 therein, and is further configured to connect to a syringe (not shown) or other medical device or fluid container. The medical device can be, for example, a patient line, vial adapter, fluid container, or infusion adapter. The syringe adapter 12 can be used to facilitate the closed transfer of fluids between the container 18, through the vial adapter 14, and into a syringe or fluid container. The syringe adapter 12 is similar to and operates in a similar manner as the adapter shown and described in U.S. Pat. No. 10,744,315, which is hereby incorporated by reference in its entirety.

The adapter 12 includes a housing 22 having a first end 24, a second end 26, and a longitudinal axis 28 extending therebetween. The housing 22 defines or includes an interior cavity 30. The first end 24 of the housing 22 of the adapter 12 may include a syringe or device attachment feature 32, such as a female luer connector, that defines a passageway 34. Although a female luer connector is shown for connection with a corresponding male luer connector of a syringe (not shown), other suitable connection arrangements may be utilized for connection to a syringe, container, or any other medical device. A cannula 36 is secured to the housing 22 and defines a proximal end 38 adjacent to or otherwise in fluid communication with the passageway 34, and the cannula 36 defines or includes a distal end 40 within the cavity 30 of the housing 22 extending in the direction of the second end 26. The syringe adapter 12 defines or includes an opening 41 at the second end 26 that is sized and shaped to receive at least a portion of the vial adapter 14 therethrough.

The adapter 12 further includes a seal arrangement 42 positioned within the housing 22 and configured to move within the interior cavity 30 in an axial direction along the longitudinal axis 28. The seal arrangement 42 may include one or more collets or other structural framing components and/or one or more membranes, barriers, or other fluid impermeable features to prevent or substantially reduce fluid flow through the distal end 40 of the cannula 36 until the adapter 12 is coupled to the vial adapter 14 and/or is otherwise prepared for fluid transport.

The adapter 12 may include a drive member 44 that biases the axially-movable seal arrangement 42 towards a first position adjacent to or in proximity to the second end 26 of the housing 22. The drive member 44 may include a spring or other biasing means configured to provide the features described herein. Upon an application of an axial force, the biasing of the drive member 44 is overcome, and the seal arrangement 42 can be moved axially in a direction towards the first end 24 of the housing 22, such that the distal end 40 of the cannula 36 pierces or otherwise passes through the seal arrangement 42, as described further herein. Upon disengagement or release of the axial force against the drive member 44, the seal arraignment 42 is biased back to its original position towards the second end 26 of the housing 22, thereby positioning the distal end 40 of the cannula 36 within or proximal to the seal arrangement 42. Such an arrangement shields the distal end 40 of the cannula 36 to prevent accidental needle sticks and also prevents the leakage of any fluid during transfer of fluids when using the syringe adapter 12.

The syringe adapter 12 may include or define one or more features to releasably and securely engage the vial adapter 14 to prevent or significantly reduce the likelihood that the syringe adapter 12 shifts, rotates along an axis oblique to the longitudinal axis 28, or otherwise moves such that leakage occurs along the fluid path between the syringe adapter 12 and the vial adapter 14 when assembled together. For example, the syringe adapter may include one or more locking elements 46 extending radially from the exterior of the housing 22 at, adjacent, or in proximity to the second end 26 of the housing 22. The locking elements 46 may extend from the housing 22 substantially transverse to the longitudinal axis 28, and may be radially spaced around the perimeter or circumference of the second end 26 of the housing 22. In the illustrated embodiment, there are two locking elements 46 spaced equidistant (e.g., at 180 degrees apart) around the second end 26 and opening 41 of the housing 22. Variations in the quantity and radial placement of the locking elements 46 are contemplated and may be modified for specific applications. Each of the one or more locking elements 46 may include or define a trapezoidal or frustoconical cross section along the longitudinal axis to aid in secure fitment with the vial adapter, as described further herein. The one or more locking elements 46 may be constructed form one or more polymers, metals, or other materials differing from or the same as the housing 22 in order to provide sufficient rigidity and strength under forces experienced during use of the system 10.

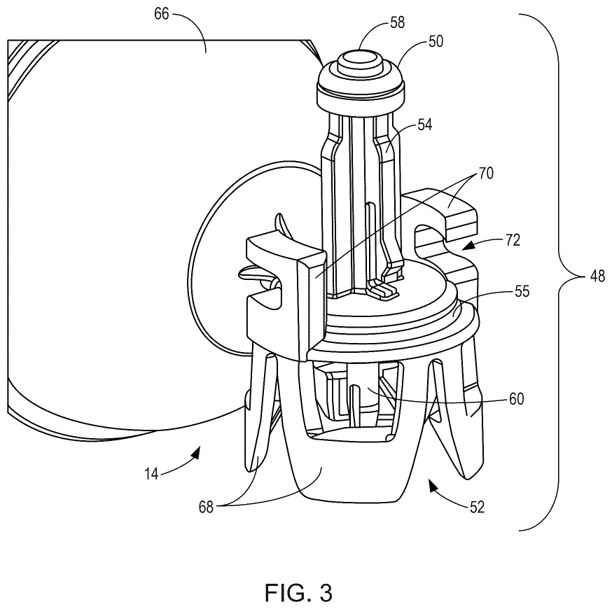

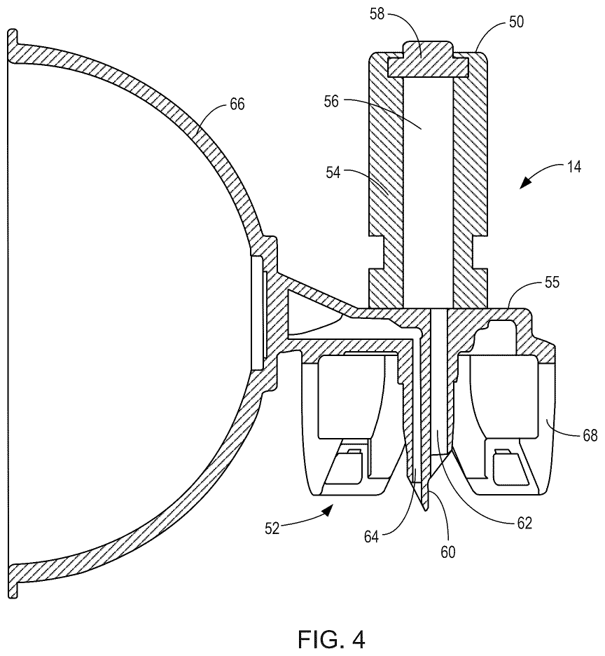

Referring now to FIGS. 3 and 4, the vial adapter 14 includes a body 48 having a first end 50 and a second end 52. The first end 50 is configured to engage with or otherwise be assembled with the second end 26 and/or interior cavity 30 of the syringe adapter 12. The body 48 of the vial adapter 14 may include an elongated segment 54 that extends to and/or defines the first end 50, where the segment 54 defines or includes a lumen 56 therein extending substantially parallel to the longitudinal axis 28. The elongated segment 54 may have a narrower or smaller diameter or width compared to a base or flange component 55 constituting a middle or intermediate segment between the first and second ends 50, 52 of the body 48. The vial adapter 14 may include a membrane 58 at or adjacent to the first end 50 of the vial adapter 14. The membrane 58 seals the lumen 56 at or near the first end 50 of the vial adapter 14 until pierced for secure transfer of fluids as described further herein. The membrane 58 may include one or segments of fluid impermeable material, layers, coatings or otherwise to prevent or substantially reduce fluid flow through the lumen 56 at the first end 50. The first end 50 of the vial adapter 14 is positionable through the opening 41 in the second end 26 of the housing 22 such that at least a portion of the vial adapter 14 is positioned within the interior cavity 30 of the housing 22. The vial adapter 14 may be positioned within the housing 22 such that the membrane 58 of the vial adapter 14 abuts or is otherwise adjacent to the seal arrangement 42 of the adapter 12.

The second end or segment 52 of the vial adapter 14 is configured and operable to engage with the first container 18 (such as a vial, jar, or the like) for secure fluid transfer from the container 18, through the vial adapter 14, to the syringe adapter 12, and towards a container or device coupled to the first end 24 of the syringe adapter 12. The second end 52 includes a spike member 60 that is configured to pierce the vial or container 18. The spike member 60 defines a fluid channel 62 and a vent channel 64. The fluid channel 62 is connected to or otherwise in fluid communication with the lumen 56 extending through the elongated segment 54. The vent channel 64 is in fluid communication with a pressure equalization device 66, such as a balloon-type or bellows-type pressure equalization arrangement, although any suitable pressure equalization arrangement 66 may be utilized. The pressure equalization device 66 is configured to maintain the pressure within the vial 18 during fluid transfer to prevent the vial 18 from being pressurized or place in a vacuum. The second end 84 of the vial adapter 14 also includes one or more vial attachment elements 68 to secure the vial adapter 14 to the vial 18 or other container. The vial attachment elements 68 may include on or more tabs, snaps or other mechanical components that matably secure the adapter 14 to the vial 18 for fluid transfer.

The vial adapter 14 may define or include one or more features to releasably engage the syringe adapter 12 while providing axial and rotational interlocking during fluid transfer to reduce or prevent leaks. For example, the vial adapter 14 may include or define on or more retention elements 70 on or extending from the body 48 that matably connect with or receive at least a portion of the locking elements 46 of the syringe adapter. Each of the one or more retention elements 70 may be mounted on or extend from the body 48 of the vial adapter 14 and constitute a bracket, catch, or other structure defining a slot 72 sized and shaped to receive a locking element 46 therein. The slot 72 may include or define tapered dimensions in one or more planes to provide secure fitment and positioning of the locking element 45 within the slot 72. In the illustrated example, the slot 72 extends substantially transverse to the longitudinal axis such that the syringe adapter 12 and the locking elements can be rotated about the longitudinal axis to engage the locking elements 46 with the retention element 70, as described herein. In the illustrated embodiment, there are two retention elements 70 spaced equidistant (e.g., at 180 degrees apart) around the body 48 of the vial adapter to matably engage the locking elements 26, and the retention elements 70 are sufficiently spaced apart laterally to allow the housing 22 of the syringe adapter 12 to fit therebetween when assembled to the vial adapter 14. Variations in the quantity and placement of the retention elements 70 about the body 48 of the vial adapter 14 are contemplated and may be modified for specific applications.

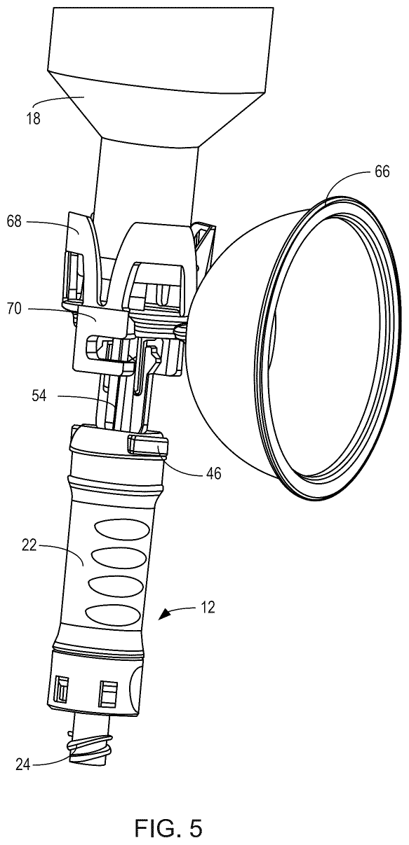

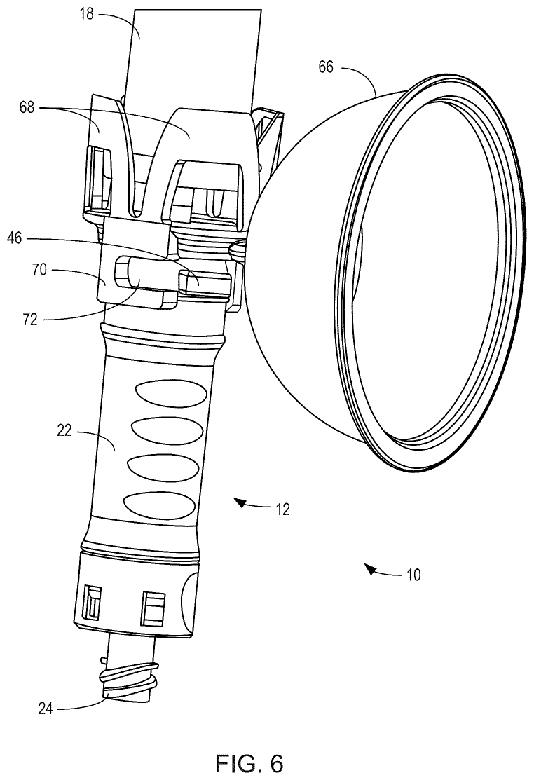

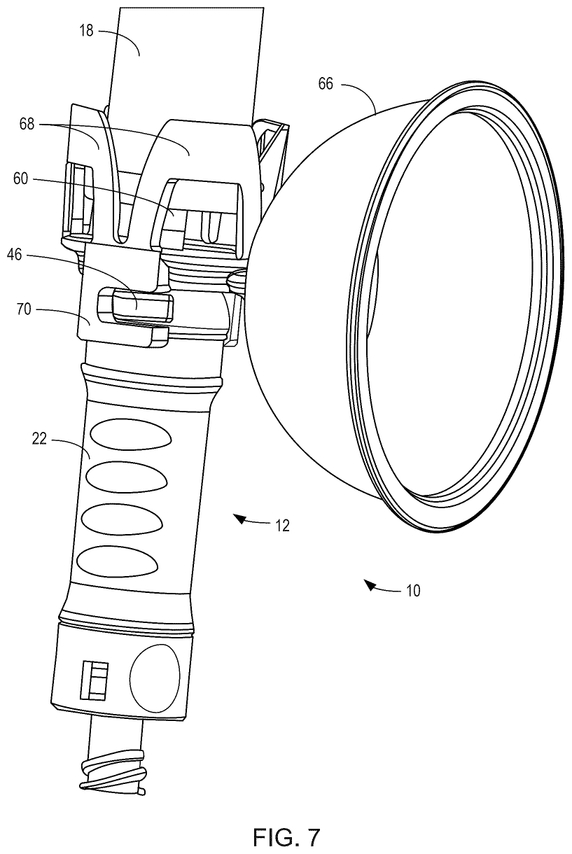

In an exemplary system assembly and use thereof, the syringe adapter 12 is releasably secured to the vial adapter 14, and fluid is transferred from the vial 18 through the vial adapter 14 to the syringe adapter and any container or device coupled downstream of the syringe adapter 12. For example, as shown in FIG. 5, the syringe adapter 12 is coaxially aligned with the body 48 of the vial adapter 14 along the longitudinal axis 28, and the locking elements 46 are radially offset from the retention elements 70. The syringe adapter 12 and the vial adapter 14 are axially moved towards each other to place at least a portion of the elongated segment 54 into the interior cavity 30 of the housing 22 of the syringe adapter 12 until the locking elements 46 are adjacent to the retention elements 70, as shown in FIG. 6. Further axial advancement of the syringe adapter 12 may be limited by the second end 26 of the housing 22 abutting a portion of the body 48 of the vial adapter at or near the base or flange 55 of the body 48. Once in the desired axial position relative to the vial adapter 14, the syringe adapter 12 may be rotated about the longitudinal axis 28 to position the locking elements 46 within the slots of the respective retention elements 72, as shown in FIG. 7.

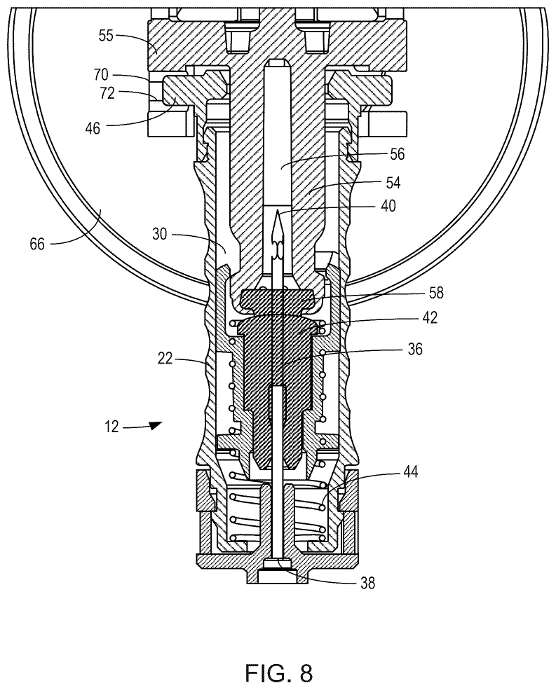

As shown in FIG. 8, the coaxial insertion and positioning of a portion of the vial adapter 14 within the cavity 30 of the housing 22 axially moves the seal arrangement 42 towards the first end 24 of the syringe adapter 12. Movement of the seal arrangement 42 causes the distal end 40 of the cannula 36 to pierce the seal arrangement 42 and the membrane 58 of the vial adapter 14, resulting in the cannula 36 being at least partially positioned within the lumen 56 of the body 48 of the vial adapter 14. As a result, the assembled syringe adapter 12 and vial adapter 14 provides a fluid flow path from the container 18 through the fluid channel 62, into the lumen 56, through the cannula 36, and out of the first end 24 of the syringe adapter 12, which may be attached to a syringe, fluid line, or other device. With the locking elements 46 secured in the slots 72 of the retention elements 70, movement between the syringe adapter 12 and vial adapter 14 that could compromise the fluid path integrity is restricted. For example, unintentional pivoting or rotation oblique to the longitudinal axis and/or lateral displacement between the two adapters that could displace the positioning of the membrane 58 with respect to the seal arrangement 42 and/or the cannula 36 and thus compromise the integrity of the fluid flow path is substantially reduced or eliminated.

Other features and details regarding the devices and methods disclosed herein are provided in U.S. Pat. No. 11,147,958, which is hereby incorporated by reference in its entirety.

It will be appreciated by persons skilled in the art that the present disclosure is not limited to what has been particularly shown and described herein above. In addition, unless mention was made above to the contrary, it should be noted that all of the accompanying drawings are not to scale. Of note, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the examples disclosed herein are not necessarily exclusive of each other and may be included in a variety of different combinations or configurations without departing from the scope and spirit of the disclosure. A variety of modifications and variations are possible in light of the above teachings without departing from the scope and spirit of the disclosure, which is limited only by the following claims.

What is claimed is:

1. A system for transferring fluids, comprising:
a syringe adapter comprising:
a housing defining a first end, a second end, and a longitudinal axis extending from the first end to the second end;
a cannula positioned within the housing;
a seal arrangement movably positioned within the housing;
at least one locking element extending from the housing; and
a vial adapter releasably attachable to the syringe adapter, the vial adapter comprising:
a body with a first end and a second end opposite the first end, wherein the first end is positionable within the housing of the adapter and the second end is configured to attach to a container; and
at least one retention element coupled to the body, the retention element configured to rotationally receive the at least one locking element to restrict axial movement of the syringe adapter.

2. The system of claim 1, wherein the body of the vial adapter defines a lumen therein, and the vial adapter further comprises a membrane adjacent the first end of the body in fluid communication with the lumen.

3. The system of claim 2, wherein the membrane is positionable adjacent the seal arrangement of the syringe adapter when the first end of the vial adapter is positioned within the housing of the syringe adapter.

4. The system of claim 3, wherein at least a portion of the cannula is positioned within the lumen when the membrane is positioned adjacent the seal arrangement of the syringe adapter.

5. The system of claim 2, wherein the second end of the vial adapter includes a spike member having a fluid channel therein in fluid communication with the lumen.

6. The system of claim 5, wherein the spike member includes a vent channel in fluid communication with a pressure equalization device.

7. The system of claim 6, wherein the pressure equalization device includes a balloon.

8. The system of claim 1, wherein the syringe adapter further comprises a spring configured to bias a position of the seal arrangement within the housing.

9. The system of claim 1, wherein the at least one locking element extends from the housing transverse to the longitudinal axis.

10. The system of claim 9, wherein the at least one locking element is adjacent the second end of the housing, and the first end of the housing includes a luer connector defining a passageway therethrough in fluid communication with the cannula.

11. The system of claim 9, wherein the at least one locking element includes a plurality of locking elements spaced equidistant around a circumference of the housing.

12. The system of claim 1, wherein the at least one retention element defines a slot transverse to the longitudinal axis.

13. The system of claim 1, wherein the at least one retention element includes a plurality of retention elements spaced equidistant around a circumference of the body, and wherein at least a portion of the housing of the syringe adapter is positionable between the plurality of retention elements.

14. A system for transferring fluids, comprising:
a syringe adapter comprising:
a housing defining a first end, a second end, and a longitudinal axis extending from the first end to the second end;
a cannula positioned within the housing;
a seal arrangement movably positioned within the housing;
a plurality of locking elements extending from an exterior of the housing transverse to the longitudinal axis; and
a vial adapter releasably attachable to the syringe adapter, the vial adapter comprising:
a body with a first end and a second end opposite the first end, wherein the first end is positionable within the housing of the adapter and the second end is configured to attach to a container;
the body defining a lumen therein;
a spike member at the second end, the spike member having a fluid channel in fluid communication with the lumen; and
a plurality of retention elements coupled to the body, each retention element defining a slot therein configured to rotationally receive at least one of the plurality of locking elements and restrict axial movement of the syringe adapter.

15. The system of claim 14, wherein the vial adapter further comprises a membrane sealing at least a portion of the lumen, and wherein the membrane is positionable adjacent the seal arrangement of the syringe adapter when the first end of the vial adapter is positioned within the housing of the syringe adapter.

16. The system of claim 15, wherein at least a portion of the cannula is positioned within the lumen when the membrane is positioned adjacent the seal arrangement of the syringe adapter.

17. The system of claim 13, wherein the spike member includes a vent channel in fluid communication with a pressure equalization device.

18. The system of claim 14, wherein the plurality of locking elements are spaced equidistant from each other adjacent the second end of the housing.
